(19) United States
(12) Patent Application Publication
(10) Pub. No.: US 2006/0117411 A1
(43) Pub. Date: Jun. 1, 2006

(54) Enhancing plant pathogen resistance via increasing BiP levels

(76) Inventors: Jurgen Denecke, Leeds (GB); Edith Jelitto, Klosterneuberg (AT)

Correspondence Address: MYERS BIGEL SIBLEY & SAJOVEC, PO BOX 37428, RALEIGH, NC, 27627, US

(21) Appl. No.: 11/053,045

(22) Filed: Feb. 8, 2005

Related U.S. Application Data
(63) Continuation of application No. 09/868,434, filed on Jan. 10, 2002, now abandoned.

(30) Foreign Application Priority Data
Dec. 15, 1998 (GB) 9827480.6

(51) Int. Cl. A01H 1/00; A01N 37/36; C12N 5/04

(52) U.S. Cl. 800/279; 514/159; 435/419

(57) ABSTRACT

The present invention provides a method of reducing the period within which a plant's natural defence mechanism responds to attack by a plant pathogen, the method comprising causing the plant to maintain, in at least a part of the plant, a level of BiP, or a homologue thereof, which is greater than the endogenous level for said plant in non-stressful conditions. Increased BiP levels can be achieved by transformation with a nucleic acid encoding BiP or calreticulin or modifying signal transduction pathways leading to BiP induction. BiP levels can be increased above endogenous levels by over-expressing BiP or calreticulin. The invention also provides for a modified plant produced by the method of the invention.

### FIELD OF THE INVENTION

This invention relates to methods of increasing the resistance of plants to pathogen attack. The invention also provides plants modified to improve their pathogen resistance without loss of productivity/yield.

### BACKGROUND OF THE INVENTION

Plant Pathogenesis

Infection of crops by pathogens leads to significant losses in agricultural yields and the study of plant-pathogen interactions has become an important area of research Upon pathogen attack, plants are able to respond by the induction of defence mechanisms. Bacterial and fungal plant pathogens often produce and secrete a large variety of hydrolytic enzymes which break down the plant cell wall. This helps penetration of the pathogen into the plant tissue, but also causes the release of cell wall degradation products, which are taken up as nutrients. Plants have developed defence responses which utilise such products as elicitors for the production of defence related proteins to combat the pathogen and prevent further pathogen attack.

Defence Mechanisms

The defence mechanism is not restricted to the infected leaf (Ross, 1961), but may also occur in distal uninfected leaves. One example of such a process is termed systemic acquired resistance (SAR; Ryals et al, 1992). Co-ordinately with the onset of SAR, a distinct set of defence genes are induced; the so-called SAR genes. SAR genes are used as molecular markers for the pathogenesis induced defence mechanism (Ward el al, 1991). Salicylic Acid (SA) has been implicated in SAR because exogenously applied SA induces SAR and the expression of SAR genes in the absence of pathogens (Ward et al, 1991). Furthermore, transgenic plants which overexpress the enzyme salicylate hydroxylase, which converts SA to a non-active form, are unable to induce systemic SAR induction (Delaney et al, 1994; Gaffney et al, 1993).

Although SA is an important compound in establishing the pathogen defence mechanism, there are pathogens which induce the plant defence response independently of SA. Examples of these are bacterial pathogens such as Erwinia carotovora which secrete plant cell wall-degrading enzymes like pectate lyases, polygalacturonases, cellulases and proteases (reviewed by Pérombelon and Salmond, 1995). Furthermore, plants treated with the cell wall-degrading enzymes pectinase and cellulase showed a proper defence response locally as well systemically (Vidal et al, 1997, 1998) by a signal transduction cascade that is independent of SA (Vidal et al, 1997). In summary, numerous molecules including salicylic acid (SA), ethylene, jasmonates and abscisic acid (ABA) have been proposed to act as local or systemic signals leading to the accumulation of defense related proteins.

Defense related proteins can have a variety of functions, leading to several possible defense mechanisms, such as cell wall strengthening or production of anti-microbial components. These proteins have been termed pathogenesis-related (PR) proteins (reviewed by Benhamou, 1996). Some of the PR proteins have been identified as chitinases and β-1,3 glucanases, and have been shown to inhibit fungal growth (Mauch et al, 1988; Sela-Buurlage et al, 1993; Lamb et al,1993). Most of these proteins are synthesised by the rough endoplasmic reticulum (ER) to be secreted or deposited in vacuoles. Most of the PR genes are induced relatively slowly, with detectable changes in gene expression ranging from several hours to days after the original stimulus.

The possible Role of the Endoplasmic Reticulum in Defense Responses

It has been reported that plant-pathogen interactions lead to increased endoplasmic reticulum (ER) chaperone expression including endoplasmin, the lumenal binding protein (BiP), protein disulfide isomerase (PDI) and calreticulin (Walther-Larsen et al, 1993; Denecke et al, 1995). This could be due to the fact that vegetative plant tissues such as leaves have a low activity in protein synthesis and transport by the secretory pathway and contain low levels of ER chaperones. ER chaperones would be required for the efficient synthesis of PR proteins on the rough ER, and the lumenal binding protein (BiP) is likely to be the most important of these.

Structural and Functional Characterisation of BiP

Several proteins have been identified in various components of eukaryotic cells as part of a polypeptide folding machinery. These so-called ‘molecular chaperones’ bind to nascent polypeptide chains and transiently stabilise the unfolded state until correct folding is accomplished.

Heat shock proteins of the hsp70 family and their related constitutive analogues form a group of molecular chaperones that are highly conserved among different eukaryotes. They have a highly conserved N-terminal ATP binding domain and a more variable protein binding domain. The lumenal binding protein (BiP), identified in various mammals and yeasts, is a member of the hsp70 family that accomplishes its function in the lumen of the endoplasmic reticulum (ER).

cDNAs of the tobacco homologue of the lumenal binding protein (BiP) have been cloned (Denecke et al., 1991). BiP differs from other family members in the presence of an N-terminal signal sequence that is required for the co-translational translocation of proteins through the ER membrane. Another specific feature is the C-terminal tetrapeptide Lys-Asp-Glu-Leu (KDEL) for mammals and His-Asp-Glu-Leu (HDEL) for yeasts that serves as a general retention signal for soluble reticuloplasmins in the ER lumen.

This structural homology to the hsp70 family, estimated at 50 to 60% across the entire protein, provides the means for a structural assay for BiP and its homologues in plants. BiP is also essential for cell viability: loss of BiP function blocks translocation of secretory proteins in yeast (Vogel et al., 1990) which suggests that the gene product plays a constitutive role both in protein import into the lumen of the ER and in the subsequent maturation steps in vivo. This provides means for a functional assay whereby a ‘suspect’ gene is introduced into a BiP deficient yeast cell. BiP or any homologue thereof can be identified by viability of the cell.

### STATEMENT OF INVENTION

As indicated above, the present invention relates to the regulation mechanism of ER chaperone gene expression during the plant defence response. The present invention is based on the surprising discovery that ER chaperones such as BiP are very rapidly induced prior to the induction of PR gene expression. Moreover, the levels of BiP in the ER lumen have to rise above a threshold level before PR gene induction can occur.

Increased BiP Levels Lead to an Accelerated Defence Response

BiP is induced locally and systemically and well before the induction of defence related genes is initiated. Limiting the amount of free BiP (i.e. by superimposing the unfolded protein response (UPR)) during the pathogen response represses PR gene induction, suggesting that PR gene induction requires availability of BiP. In contrast, constitutive overproduction of BiP leads to an accelerated defence response without any deleterious effect on the viability of the plant. Crucially, the overproduction of BiP alone is not sufficient to cause induction of PR genes. This could have numerous economic benefits because an accelerated defense response would not imply a waste of energy in the absence of pathogens. In comparison, treatment of crops with SA or derivatives implies constitutive production of defence-related proteins even when pathogens are absent, thus wasting energy and reducing yields.

According to the present invention there is provided a method of reducing the period within which a plant's natural defence mechanism responds to attack by a plant pathogen, the method comprising, causing the plant to maintain in at least a part of the plant a level of BiP, or a homologue thereof, which is greater than the endogenous level for said part of the plant in non-stressful conditions.

Preferably the maintained level of BiP or homologue thereof, is at least three times said endogenous level, more preferably the maintained level is at least five times said endogenous level. The present invention thus provides a modified plant which maintains, in at least a part thereof, a level of BiP, or a homologue thereof, greater than the level maintained in said part by an unmodified plant of the same species in non-stressful conditions. Although the word ‘species’ is used for the purposes of this patent application, it is appreciated that the modified plant is now of a different genetic make-up and is therefore not strictly classified within the same generic subordinate.

Preferably overproduction of BiP is directed at the leaf although any part of the plant may be targeted. The stem for example is highly susceptible to bacterial infection. Plants that overproduce BiP constitutively, either via direct overproduction or indirect induction using other methods, show an accelerated plant defence response. The method of the present invention thus harnesses the plants own defence mechanism, but since the response is faster, pathogens would have less time to establish themselves. A higher resistance to pathogen infection would be the result, avoiding the need to spray plants with signalling molecules such as SA or SA analogues to induce SAR. The advantage would be that plants only produce the defence related proteins when needed (during pathogen attack) and not constitutively (thus wasting energy).

BiP overproducers could however be used in combination with treatment or SA or SA analogs, but with lower concentrations of these signalling molecules, thus reducing the costs of spraying a field. This may be more effective in protecting the plant, but it would still imply a constitutive production of defence related proteins, possibly reducing the yield of a field.

Methods to Increase BiP Levels in Plants

Numerous methods may be employed to achieve high levels of BiP in plants. The following examples are illustrative and should not be construed as limiting the scope of the present invention.

The maintained level may be effected by over expression of BiP, or a homologue thereof, by means of a chimeric gene containing a strong constitutive promoter, a coding region for BiP or a homologue thereof and a 3′ untranslated end containing a stop sequence such as a polyadenylation signal.

Alternatively the maintained level may be effected by over expression of calreticulin, or a homologue thereof, by means of a chimeric gene containing a strong constitutive promoter, a coding region for calreticulin or a homologue thereof and a 3′ untranslated end containing a stop sequence. High levels of calreticulin will induce high levels of BiP.

Another possibility is over expression of the ATPase domain of BiP, or a homologue thereof, and an ER retention signal, by means of a chimeric gene containing a strong constitutive promoter, a coding region for the ATPase domain of BiP, or a homologue thereof, and for an ER retention signal and a 3′ untranslated end containing a stop sequence. This will induce BiP production.

The maintained level of BiP may also be effected by modifying signal transduction pathways leading to BiP induction. This may be achieved through genetic engineering.

The structural and functional homology of the hsp70 family and their related constitutive analogs means that any other member of the family could be used in place of BiP to achieve the objective of accelerated plant defence response against pathogen attack. For the purposes of this patent application the term BiP includes any homologue thereof which has a significant degree of structural or functional similarity. It is also appreciated that accelerated response to pathogen attack might be achieved through increased expression of the endogenous BiP gene. High levels of BiP transcripts, detected by Northern blotting, would serve as evidence for the fact that engineering techniques have been employed. Synthetic BiP might also be applied. Possible methods of detection in this instance include producing monoclonal antibodies to highly conserved regions of the molecule.

According to a further aspect of the invention there is a modified plant or plant cells with a level of BiP, or a homologue thereof, which is greater than the endogenous level for said part of the plant in non-stressful conditions produced by the method of the present invention.

### BRIEF DESCRIPTION OF THE DRAWINGS

FIG. 1: A) Local and systemic expression of BiP and 5-β1,3-glucanase in response to cell-wall degrading enzymes (CDE). The local response was determined by treating one leaf from each tobacco plant with CDE followed by collecting the leaves after different time points (hours). Untreated leaves were collected from the same plant to measure the systemic response. 20 plants were used per time point for extracting RNA. Loading differences were analysed by hybridising the same filters with a probe against tobacco ribosomal RNA. The panels show mRNA levels of the lumenal binding protein (BiP), β-1,3-glucanase (Glu) and a riboprobe (Ribo) B) As above but instead of RNA, proteins were extracted and analysed by protein gel blots.

FIG. 2: Northern blot analysis of BiP and Hel transcripts in Arabidopsis thaliana ecotype Nössen WT and SA non-responsive (sail) mutant after treatment with CDE. Plants were mock-infected (H₂O) or treated with CDE for 6 hours wbereafter total mRNA was extracted from the treated leaves. A ribosomal RNA probe (Ribo) was used as an internal control for gel loading differences.

FIG. 3: Local and systemic analysis of BiP and β-1,3-glucanase expression during CDE infection in tobacco samson WT and NahG mutant. Plants were inoculated with CDE for 6 and 24 hours after which RNA was extracted from the treated (local) and its opposite non-treated (systemic) leaves. Loading differences were analysed using a ribosomal probe (Ribo).

FIG. 4: Effect of tunicamycin on local and systemic induction of BiP and β-1,3-glucanase expression. Tobacco SR1 leaves were inoculated with H₂O cell-wall degrading enzymes or tunicamycin (0.02 mM). Total RNA was extracted from the treated leaf (local) and its opposite leaf (systemic) after 3 and 8 hours of treatment. Equal loading of the RNA was analysed by hybridising the filters with a probe against ribosomal RNA.

FIG. 5: Expression of BiP and β-1,3-glucanase (Glu) transcripts in tobacco SR1 WT and BiP overproducing plants (89). RNA was extracted from the treated (local) leaves after 6 hours of CDE treatment and Northern blot analysis was performed. The amount of corresponding RNA in the different samples was analysed by hybridising with a ribosomal probe.

FIG. 6: RNA gel blots of total mRNA was isolated from WT SR1 protoplasts incubated in the presence (+) or absence (−) of tunicamycin (20 μg/mL) for 2.5 hrs. Blots were probed with a BLP4 probe detecting both endogenous and introduced BLP4 transcripts (BLP4), a β-1,3-glucanase probe (Glu) or a ribosomal RNA probe (Ribo) to control for loading differences.

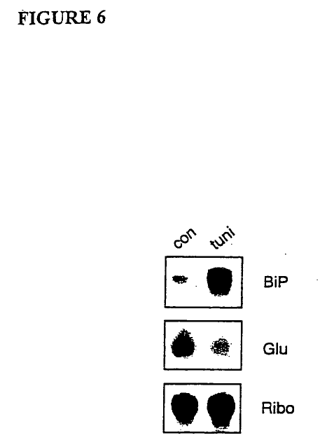

FIG. 7: (A) Time dependent induction of BiP and PR1 mRNA in response to SA. Tobacco SR1 plants were sprayed with 5 mM SA and total mRNA was extracted at the indicated timepoints (hours). Filters were probed for BiP, PR1 and a probe against ribosomal RNA (Ribo) to test equal loading of the RNA samples. (B) As above but protein levels were analysed by western blotting.

FIG. 8: mRNA levels of BiP and PR1 during SA-treatment in the presence (light) and absence (dark) of light Total RNA was extracted from tobacco SR1 plants treated with 5 mM SA and incubated in the presence and absence of light at the indicated timepoints (hours). Probes for BiP, PR1 and a ribosomal probe (Ribo) were used.

FIG. 9: Effect of cell wall-degrading enzymes (CDE) on SA induction of BiP and PR1 expression. Accumulation of BiP transcripts in tobacco SR1 plants treated with 5 mM SA in the presence (+CDE) or absence (−CDE) of cell wall-degrading enzymes was analysed. Samples were collected 6 and 16 hours after treatment as indicated. The control measurement (CO) was done at timepoint 0. The probes used are indicated on the left hand side.

FIG. 10: RNA gel blots of total mRNA was isolated from WT SR1 plants treated with 5 mM SA for 12 h after which leaves were incubated on MS medium in the presence (SA+tuni) or absence (SA−tuni) of tunicamycin (20 μg/mL) for 2.5 h. RNA was extracted and blots were probed with a BLP4 probe (BiP). A ribosomal RNA probe (Ribo) was used as a control for loading differences.

FIG. 11: Northern blot analysis of BiP and PR1 transcripts in Arabidopsis thaliana ecotype Nössen WT and SA non-responsive (saiI) mutant after treatment with SA (5 mM). Plants were treated for 8 and 20 hours whereafter total mRNA was extracted. The probes used are indicated on the left hand side as before. A ribosomal RNA probe (Ribo) was used as a control for loading differences.

FIG. 12: Time-dependent induction of BiP and PR1 gene expression in tobacco WT and tobacco plants expressing the BiP gene under control of a CaMV 35S promoter. WT and BiP overproducing plants (line 89) were treated with 5 mM SA, and mRNA and proteins were extracted after 6 and 24 hr of treatment. (A) Northern blot analysis of BiP and PR1 transcripts in wild-type tobacco and BiP overproducing tobacco treated with SA. Samples were collected at 0, 6 and 24 hours after treatment. The probes used are indicated on the left hand side as in FIG. 1. (B) As (A) but proteins were analysed by western blotting.

FIG. 13: Effect of tunicamycin on GUS and α-amylase activities. (A) Schematic representation of the chimeric genes and the vector pNL200. GUS, glucuronidase gene; AMY, α-amylase gene; mas, dual mannopine synthase promoter, Amp^{r}, ampicillin resistance; ori, E. coli origin of replication. (B) Cytoplasmic GUS and total α-amylase activities after electroporation of tobacco protoplasts and incubation for 20 hr in the presence (black bars) or absence (white bars) of tunicamycin (20 μg/mL). The activities are shown as percentages of the activities without tunicamycin (set to 100% in each case)

FIG. 14: The effect of altered BiP levels on the activity of α-amylase. (A) Coelectorporation experiments using pNL200 in the presence of plasmids carrying (1) a gene phosphinothricin acetyltransferase (PAT), (2) a BiP overexpression construct (BiP⁺) or (3) a BiP antisense construct (BiP^{as}). Cells were incubated for 20 hr in the presence (white bars) or absence (black bars) of tunicanycin (20 μg/mL) after which α-amylase and GUS activities were measured. The ratio of α-amylase/GUS activities is shown for two independent experiments. (B) Data from (A) presented as percentage of α-amylase activity that remains after tunicamycin treatment (20 μg/mL) in the different coelectroporation experiments. Note the almost full recovery to 100% in cells cotransfected with the BiP overexpression construct.

FIG. 15: Levels of transcripts encoding secretory proteins diminish during tunicamycin induced ER stress. Steady state levels of BiP and β-(1,3)-glucanase transcripts. Tobacco protoplasts were incubated in the presence of tunicamycin (20 μg/mL). Samples were taken for RNA preparation at the indicated times (min). Equal loading of the samples was detected by hybridising the same Northern with a Ribo RNA probe.

FIG. 16: BiP overexpression abolishes the reduction of β-(1,3)-glucanase mRNA levels during tunicamycin-induced ER stress. (A) Transcript levels of BiP and β-(1,3)-glucanase genes in wild type (WT) and BiP overexpressing (89) protoplasts. Tobacco protoplasts were incubated in the absence (−) or presence (+) of tunicamycin (20 μg/mL). Samples were taken for RNA preparation after 2.5 h of incubation. Equal loading of the samples was detected by hybridising the same Northern blot with a Ribo RNA probe. (B) Transcript levels of BiP and β-(1,3)-glucanase genes in wild type (WT) and BiPΔHDEL overexpressing (801) protoplasts. Samples were treated as described under A.

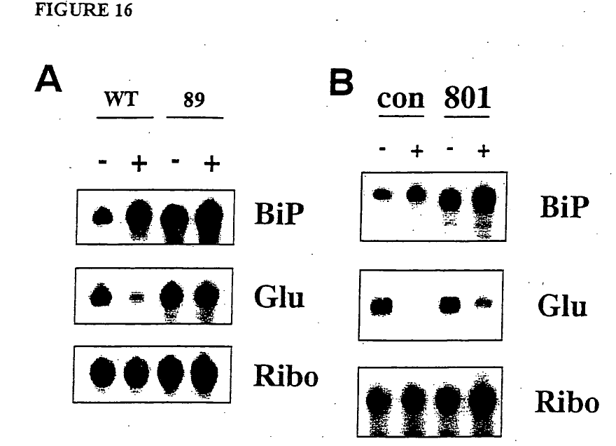

FIG. 17: Model describing the SA-mediated induction of PR genes and BiP. Both BiP and PR genes are synthesised by the rough ER. A branched signal transduction pathway leads to SA-mediated induction of BiP and PR genes with the branch point being upstream of saiI. BiP induction is faster than PR induction, and the BiP protein levels in the ER have to reach a threshold level before PR gene induction is initiated. This control acts on the saiI dependent pathway either upstream or downstream of saiI.

### DETAILED DESCRIPTION OF THE INVENTION

Treatment of tobacco plants with cell wall-degrading enzymes (i.e. cellulases and pectinases) leads to the induction of a subset of PR genes involved in the plant defence response (Palva et al., 1993; Vidal et al., 1998). This induction occurs both in the local treated leaf and the distal uninfected leaves. Furthermore, it has been shown that during plant-pathogen interactions there is ER lumenal gene induction (Walter-Larsen et al., 1993; Denecke et al., 1995).

The present invention is concerned primarily with the exact timing of chaperone induction in plants subjected to pathogen stress, which is simulated by treatment with cell wall-degrading enzymes (CDEs). ER chaperone gene induction occurs rapidly upon CDE treatment on a local as well as systemic level which is faster than the PR gene activation. The invention is also concerned with elucidating the role of SA and its signal transduction pathway in this fast-acting induction mechanism, as this molecule is known to play an important role in establishing the pathogen defence mechanism (Ward et al., 1991; Delaney et al., 1994; Gaffney et al., 1993).

The parallel induction of ER chaperones in the local and distal untreated leaves suggests the presence of a signal molecule or other such mechanism whereby a signal can be quickly transported throughout the whole plant. The results show that the systemic induction of ER chaperones is not triggered by a feedback signal mechanism resulting from accumulation of newly synthesised proteins (i.e. the UPR) in the local treated leaf. The early induction of BiP is likely to depend on a feedforward mechanism in which the plant prepares itself for the folding of newly synthesised PR proteins

Exogenously applied SA leads to an induced resistance to Erwinia carotovora subsp. carotovora in tobacco (Palva et al., 1994). Arabidopsis thaliana saiI mutants which are abolished in their PR1 gene expression during SA treatment due to a mutation in the SA signal transduction pathway (Shah et al., 1997) showed a normal CDE-mediated BiP induction. Furthermore, transgenic NahG plants which contain the enzyme salicylate hydroxylase that converts SA into a non-active form showed similar CDE-mediated BiP gene expression as in the WT plants. This clearly demonstrates that SA is not involved in the local and systemic BiP induction during CDE treatment.

Other molecules like ethylene and jasmonate might be involved in the rapid induction of ER chaperones as it is known that these compounds accumulate upon wounding and during pathogen attack thereby inducing a distinct set of genes which are thought to play a role in plant defence (Hyodo, 1991; reviewed by Boller, 1991; Creelman et al., 1992; Farmer et al., 1992). It has been shown that the CDE-induced defence response involves both the ethylene and signal transduction pathways (Vidal et al., in preparation). As ethylene and methyl jasmonate are both volatile, possible diffusion from the site of synthesis might occur, thereby acting in its gaseous form as the long distance signal. However, the role of both compounds in the early induction of BiP gene expression has still to be established. Therefore characterisation of BiP gene induction in ethylene-insensitive mutants of Arabidopsis would be a novel way to obtain more insight in the role of ethylene during CDE treatment.

The CDE signal transduction pathway leading to the BiP gene induction is independent of the β-1,3-glucanase signal transduction pathway as overproduction of the BiP protein does not lead to induced β-1,3-glucanase transcript levels. This suggests that early in the CDE signal transduction pathway BiP differentiates from the β-1,3-glucanase signal transduction route leading to a rapid BiP gene induction before that of PR genes. The plant therefore anticipates the need for more ER chaperones necessary for the expected PR gene transcripts encoding secretory defence proteins on the rough ER during defence reactions. The signal involved in the CDE-mediated initiation of BiP gene expression is still unknown although it is most likely to be independent of the UPR and SA-mediated signal transduction pathway.

Besides CDE treatment, SA also rapidly induces BiP genes well before the PR genes. The SA-dependent induction of BiP is distinct from that of the CDE-mediated BiP induction. The results show that the induction of BiP upon pathogen attack is not merely a consequence of the increased synthesis of proteins on the rough ER, but an early response of plant cells in order to prepare an adequate machinery for PR protein synthesis. The most crucial part of the invention concerns the contribution of the ER chaperone BiP in plant pathogen interactions and SAR.

BiP induction occurs rapidly via an SA-mediated signal which also induces SAR and PR protein synthesis, but the signal transduction pathway leading to BiP is faster and independent of PR gene activation. This process operates in tobacco as well as Aracbidopsis thaliana and is likely to be a conserved mechanism among plants. The sail mutant is incapable of SA-mediated PR1 induction but shows a normal SA-mediated rapid BiP induction. Artificially increased BiP levels do not lead to PR1 induction in the absence of SA, which demonstrates that BiP is not an earlier element of the SA-mediated signal transduction cascade leading to PR1. However, artificially increased BiP levels do have a synergistic action on the SA signal and accelerate PR1 induction to a give much faster response.

Working Model for the Role of BiP in SA-Mediated PR Gene Induction

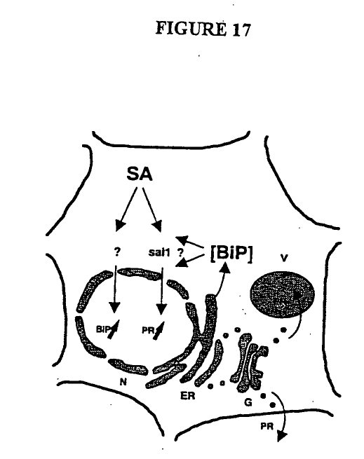

A model for the contribution of BiP in the early response of plant cells to pathogen attack is illustrated in FIG. 17. When plants are attacked by a range of pathogens. SA levels increase and mediate SAR and PR gene induction (Durner et al, 1997). Many PR proteins such as chitinases, β-1,3,-glucanases, PR1, extension and PGIP are secreted or vacuolar and are synthesised on the rough ER. Leaf mesophyll or epidermal cells in fully developed leaves do not secrete significant amounts of protein and have low levels of ER chaperones (Vitale et al, 1993). Therefore plants anticipate the need for more ER protein folding machinery to accommodate the drastically increased concentration of transcripts encoding secretory defence proteins on the rough ER during defence reactions.

SA induces BiP independently of PR genes via a branched signal transduction pathway, with the branching point being located upstream of saiI. Downstream of the branching point, elements of the signal transduction pathway leading to PR1 such as saiI (and others) must be influenced by light and cell wall-degrading enzymes, none of which have an effect on the SA-mediated BiP induction. In addition, a regulatory cross-talk between the two branches of the pathway exists to provide an additional regulatory mechanism to delay PR gene induction until BiP levels are adequate. PR gene induction is either inhibited by low BiP levels or induced by high BiP levels in combination with SA.

Such a regulatory mechanism is beneficial to the plant cell as it ensures an upregulated ER function before increased secretory protein synthesis commences in the defence response. This avoids an accumulation of translocation and folding intermediates of PR proteins in the ER due to a lack of BiP.

BiP is a key element in the early responses of plants to pathogen attack due to its requirement for the translocation and folding of proteins in the ER and to constitute an important regulatory mechanism that delays PR protein synthesis on the rough ER until the ER lumen is adequately prepared with chaperones and folding enzymes. Other ER resident proteins such as PDI and calreticulin may be important as well as they too are induced, but since BiP overexpression alone accelerates PR gene induction, BiP is the key element in the regulation. BiP should therefore be regarded as a novel target gene in early responses of plants to pathogen attack.

BiP overproducing plants have a higher viability and are more vigorous than the untransformed plants. Overexpression of BiP may have either positive effects on the ability to produce proteins, to grow faster and to resist a range of abiotic stresses such as frost, drought and salt stress.

Evidence in Support of the Model

The inhibitory effect of low BiP levels on PR gene expression is probably part of a more general regulatory mechanism to regulate protein synthesis on the rough ER. Synthesis of the secretory protein α-amylase is inhibited by ER stress which limits the level of free BiP. Artificially increased BiP levels do not show such an inhibition, demonstrating that high levels of BiP are required for efficient α-amylase synthesis under stress. This phenomenon could be related to the retardation of PR gene induction until sufficient BiP molecules are available. Further results suggest that a novel negative pathway leads to a specific reduction of transcript levels corresponding to genes encoding secretory proteins, which means proteins synthesised by the rough ER.

Overexpression of BiP could thus have additional benefits besides increased pathogen resistance, for example in the production of secretory proteins in general (of which PR proteins are merely a subset).

Our experiments have shown that BiP over-producers exhibit resistance to the bacteria causing soft rot in potatoes (Erwinia), in that the bacteria has a reduced division rate in wounded parts of BiP over-producers and so infection is reduced as compared to controls. In the field, where infection usually starts with just one bacterium, this difference may be crucial.

Additionally, we have observed that BiP over-producers grow more rapidly, set seeds more rapidly and germinate more rapidly compared to controls, moreover cells prepared from BiP over-producing plants have a higher capacity to produce protein than control plant cells.

The following examples are provided to fully illustrate the present invention and should not be construed as limiting thereof. Details of the materials and methods used are included after the specific examples.

### EXAMPLE 1

### ER chaperone expression is induced both locally and systemically

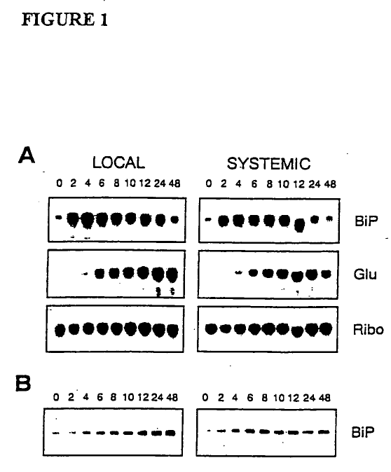

We have shown that treatment of tobacco plants with cell wall-degrading enzymes (CDE) leads to the rapid induction of β-1,3-glucanase and other PR genes, both locally and systemically (Vidal et al, 1997). We have now repeated these experiments and monitored the ER chaperone BiP as well as β-1,3-glucanase, which was the most rapidly induced PR protein in this experimental system (Vidal et al, 1997). We used a commercial preparation of fungal CDEs instead of custom made Erwinia-derived hydrolases to increase reproducibility. One leaf from each tobacco plant was treated with CDEs and the induction of BiP gene expression was analysed in the treated (local) and untreated (systemic) leaves from the same plant. FIG. 1 shows that BiP transcripts accumulate rapidly, reaching a maximum after just 2 hours of incubation. This induction was observed locally as well systemically with the same timing and intensity. β-1,3-glucanase transcript accumulation is detectable only after 4 hours of incubation and reaches its plateau after 8 hours as described previously (Vidal et al, 1997). Whereas BiP induction is transient, β-1,3-glucanase mRNA levels continue to be high after prolonged incubation times (24-48 hours). Similar patterns were obtained for PDI and calreticulin (data not shown) indicating that other reticuloplasmins are also induced both locally and systemically. Our results clearly show that BiP gene expression is induced locally as well systemically prior to the PR gene β-1,3-glucanase. The signal involved in the systemic response of BiP gene expression must therefore be transported very rapidly from the local leaf to the distal leaves.

### EXAMPLE 2

### CDE-mediated BiP gene expression in SA insensitive mutants

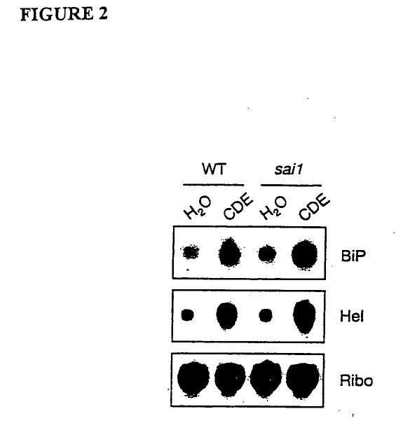

As SA was shown to induce ER chaperone expression (Denecke et al, 1995), we wanted to test if SA plays a role in the BiP gene induction during treatment with cell wall-degrading enzymes. We used as SA-insensitive mutant of Arabidopsis thaliana (saiI) which no longer shows an induction of PR1 in the presence of SA (Shah et al, 1997). WT and saiI mutants of the same ecotype were treated with cell wall-degrading enzymes and incubated for 6 hours. As a control (con), leaves were mock-infected with H₂O. Total RNA was extracted and Northern blot analysis was performed for BiP and a hevein-like protein (Hel) which is known to be induced by CDEs of Erwinia carotovora (Vidal et al, in preparation). FIG. 2 shows that WT Arabidopsis thaliana plants show a specific CDE-induced accumulation of BiP transcripts (compare with the mock infection) as in tobacco plants. In the Arabidopsis saiI mutant, the BiP gene expression exhibits exactly the same profile as seen in Arabidopsis WT plants. Transcripts of Hel were also induced in wild-type plants. This demonstrates that either the mutation in the saiI is downstream of the BiP gene induction in an SA signal transduction pathway or that SA and the saiI-dependent signal transduction pathway is not involved at all in the CDE-mediated BiP gene induction.

### EXAMPLE 3

### Both local and systemic induction of BiP and Glucanase by cell wall-degrading enzymes is SA independent

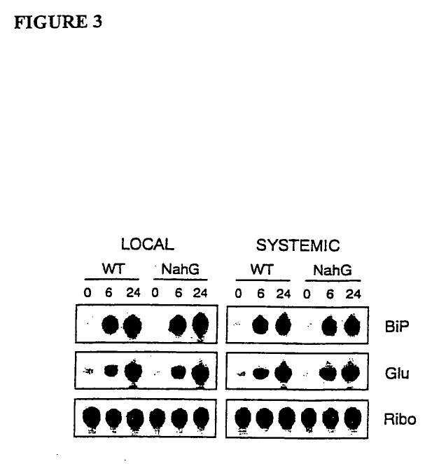

Although saiI mutants are insensitive to SA it is known that they are still able to accumulate SA upon pathogen infection (Shah et al., 1997). The previous experiment could not rule out completely the involvement of SA in the CDE-mediated BiP gene expression. To test this possibility, we used transgenic tobacco plants (NahG) that overexpress the enzyme salicylate hydroxylase which inactivates SA. It is clearly established that such plants are unable to accumulate SA (Gaffney et al., 1993). After 6 and 24 hours of CDE treatment, local and systemic leaves were harvested from unstransformed tobacco plants (WT) and NahG plants for RNA extraction. As a positive control, the expression of β-1,3-glucanase was monitored because CDE treatment will lead to its local and systemic induction independently of SA (Vidal et al., 1997). Our results clearly demonstrate that the presence of the NahG geneproduct has no influence on the CDE-mediated BiP induction (FIG. 3). This shows that SA is not involved in the signal transduction mechanism for both the local and the systemic induction of BiP in this experimental system. As expected β-1,3-glucanase was induced locally and systemically in both WT and NahG plants when treated with CDE as shown before (Vidal et al., 1997).

### EXAMPLE 4

### Systemic induction of ER chaperones is independent of the UPR in local leaves

Even though BiP induction occurs prior to β-1,3 glucanase induction, we cannot rule out that other, as yet unidentified defence related proteins are induced more rapidly and perhaps before BiP in our experimental system. If this were the case, it would still be possible that the rapid induction of BiP is the result of a feedback mechanism due to ER stress resulting from the increased synthesis of proteins on the rough ER, the unfolded protein response (UPR). In addition, a unique feature of the plant ER is its continuity through the entire plant through the numerous plasmodesmata. We thus wanted to test if a UPR triggered locally could result in a systemic UPR in cells that do not suffer from ER stress. We treated one leaf of a tobacco plant with the drug tunicamycin, which inhibits N-glycosylation of proteins in the ER and causes the accumulation of malfolded proteins and the UPR (Kosutsumi et al., 1988; Shamu et al., 1997). As a negative control, tobacco leaves were mock-infected with H₂O to examine possible induction of reticuloplasmin gene expression upon wounding whereas the positive control was the infection with CDEs. Total RNA was extracted from the treated (local) leaves and the distal untreated (systemic) leaves after 3 and 8 hours of incubation.

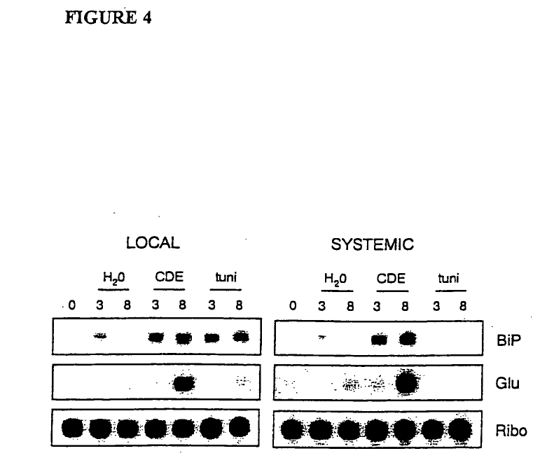

BiP gene expression during mock-infection shows a slight induction after 3 hours of treatment in the local and systemic. This induction is shown to be transient and BiP mRNA levels return to their basic steady state levels after 8 hours (FIG. 4). β-1,3-glucanase mRNA levels, however, do not increase at all during the mock-infection, confirming a minor influence of the wound response in our experimental system. Treatment of tobacco leaves with cell wall-degrading enzymes shows a local and systemic induction of BiP and β-1,3-glucanase genes, with the BiP induction being the fastest response as seen in FIG. 1. When tobacco leaves are treated with tunicamycin a strong increase of BiP mRNA levels is observed after 3 and 8 hours in the treated leaf, but not the systemic leaf. This shows that the UPR alone cannot constitute a systemic signal to induce BiP in plants. The sytemic signal must thus be a novel compound that has yet to be identified. In addition, the expression of β-1,3-glucanase was neither locally nor systemically induced upon tunicamycin treatment, demonstrating that the UPR is not involved in the production of defence related proteins either. We postulated that BiP gene induction occurs via a feedforward mechanism in which the plant anticipates the need for more ER chaperones for the folding of newly synthesised PR proteins.

### EXAMPLE 5

### Overexpression of BiP is not sufficient to trigger the induction β-1,3-glucanase

The more rapid induction of BiP compared to β-1,3-glucanase and the otherwise similar expression profiles could suggest that BiP is an element of the signal transduction cascade leading to the defence genes. To test this, we used transgenic plants which overproduce BiP under the control of the strong constitutive Cauliflower Mosaic Virus (CaMV) 35S promoter. These plants show a 142-fold increase in BiP transcript levels and a 5-fold increased BiP steady state protein level. If BiP were part of the signal transduction cascade leading to the target gene β 1,3-glucanase, BiP overexpression alone should trigger β-1,3-glucanase gene induction.

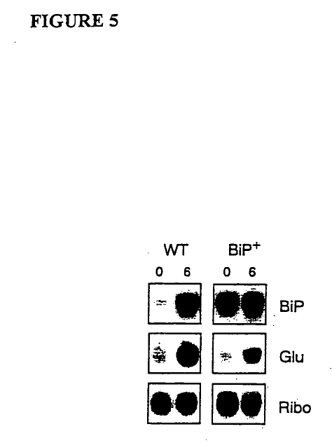

RNA was extracted from untreated (t=0) and CDE-treated (6 hours) plants after which BiP and β-1,3-glucanase are detected. FIG. 5 shows that BiP overproduction alone does not lead to the induction of β-1,3-glucanase (compare lanes 0 with each other). Otherwise, β-1,3-glucanase was induced after 6 hours of CDE treatment in both the WT and BiP overproducing plants (lane 6 WT and 89). The figure also illustrates the higher BiP mRNA levels in BiP overproducing plants (compare lanes 0 with each other). The data clearly demonstrate that high BiP protein levels do not replace the signal which leads to the β-1,3-glucanase gene induction during CDE treatment. Thus, BiP is not part of the signal transduction pathway leading to β-1,3-glucanase.

### EXAMPLE 6

### The unfolded protein response is additive to the CDE response of BiP and inhibits the expression of β-1,3glucanase.

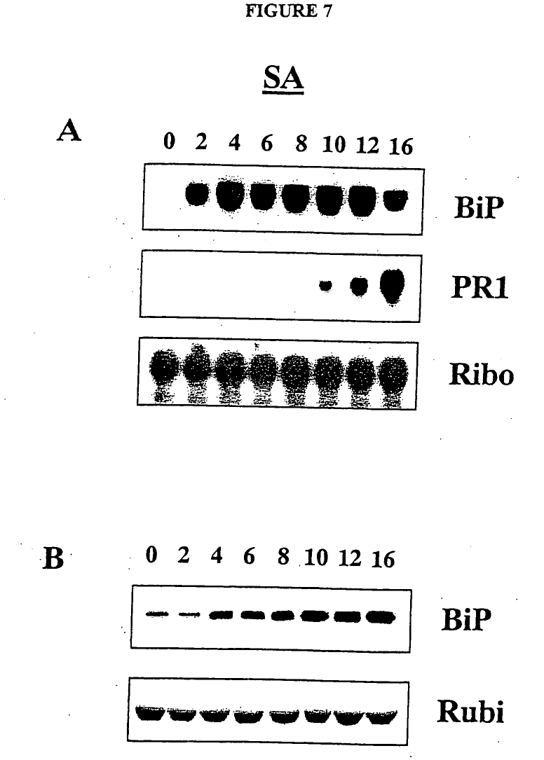

To compare the CDE- and UPR-mediated induction of BiP, we wanted to test if both stimuli are additive. For this purpose, we prepared protoplasts which are known to exhibit induced levels of β-1,3-glucanase (Denecke et al., 1995). This is not surprising as protoplasts are prepared with CDEs. These protoplasts were then treated with tunicanycin, to superimpose the UPR onto the CDE response. FIG. 7 shows that both stimuli are additive, exhibited by a further induction of BiP by tunicamycin. This suggests that both mechanisms are different. Interestingly, β1,3-glucanase expression is inhibited by tunicamycin. The additional ER stress could trap BiP in malfolded protein complexes, thus making it unavailable to promote PR protein synthesis on the rough ER. The results suggested that although BiP induction alone is not sufficient to trigger PR protein synthesis, sufficient BiP levels are required to promote PR gene expression.

### EXAMPLE 7

### SA induces BiP mRNA levels prior to PRI

It has been shown that SA is involved in the induction of PR genes (reviewed by Malamy and Klessig, 1992) and that this signal molecule also induces the expression of ER chaperones (Denecke et al, 1995). We wanted to investigate whether ER chaperone induction is a consequence of the high synthesis rates of PR genes and used BiP as a model system. Tobacco plants were sprayed with SA (5 mM) and the exact timing of the SA-mediated induction of PR-gene PRI was compared to that of BiP. PR1 was chosen as a representative marker for SA-mediated induction of PR genes as its response to SA is faster and more pronounced than that of acidic chitinase, basic chitinase and basic β-1.3 glucanase (Vidal et al, 1997). FIG. 7A shows that the BiP mRNA levels are induced after 2 hours of treatment with SA and reach a plateau after 4 hours. In contrast SA-induced transcription of PR1 starts only after 6-8 hours and continues to accumulate upto 16 hours after treatment. At this time-point, BiP mRNA levels start to decrease again. We have shown previously that PR1 continuous to accumulate until 48 hours after SA treatment using the same experimental system (Vidal et al, 1997). Similar patterns where obtained for PDI and calreticulin (data not shown) indicating that other reticuloplasmins are upregulated as well. The data also show that BiP expression during SA-treatment is unlikely to be triggered by a feedback mechanism resulting from the presence of newly synthesised and perhaps malfolded or partially folded PR proteins in the ER. BiP protein levels were also shown to increase (FIG. 7B), with a significant increase noticeable after 4-6 hours of induction.

### EXAMPLE 8

### SA-mediated induction of BiP is not light dependent

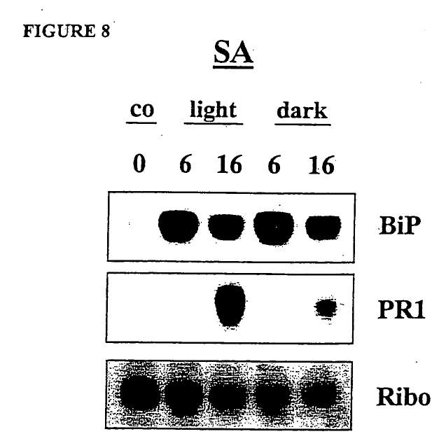

To investigate further similarities and differences in the SA-mediated induction of BiP and PR genes, we tested if the presence of light is required for the induction. The rationale for this experiment was derived from the observation that SA inhibits catalase, resulting in an increase of active oxygen species in the plant, which would then induce PR genes such as PR1 (Chen et al, 1993). Despite the fact that the inhibition of catalase activity alone is not the key route by which PR1 is induced during SA-treatment (Chamnongpol et al, 1996), a cooperative interaction of SA and H₂O₂ might lead to the strong induction of PR1 (Leon et al, 1995, Chen et al, 1995). Since photorespiration is a major source of hydrogen peroxide in plant cells, SA would be a less effective inducer during darkness. Tobacco plants were thus sprayed with SA (5 mM) and incubated in the presence and absence of light for 6 and 16 hours. In the absence of light, BiP showed hardly any reduction in the accumulation of mRNA transcripts during SA-treatment whereas PR1 induction was almost completely abolished (FIG. 8). These data demonstrate that the SA-mediated induction of BiP differs from that of PR1. The data also confirm that BiP mRNA levels increase transiently and a significant decrease in mRNA levels is detectable after 16 hours of treatment (see also FIG. 7).

### EXAMPLE 9

### Cell wall-degrading enzymes antagonise SA-mediated induction of PR1 but not BiP

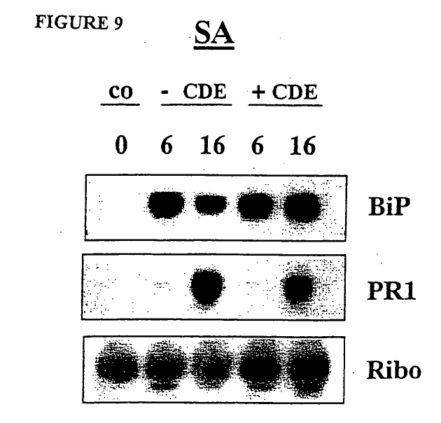

We have shown that the SA-induced expression of PR1 is inhibited in a concentration dependent fashion by the presence of Erwinia carotovora culture filtrate containing cell wall-degrading enzymes (Vidal et al, 1997). Plant cell wall-degrading enzymes antagonise the effect of SA-dependent PR-gene expression via an unknown mechanism, and we wanted to test if cell wall-degrading enzymes had a similar antagonistic effect on SA-mediated BiP expression. Tobacco plants were therefore treated with a solution of 5 mM SA with or without cell wall-degrading enzymes (0.2% macerozyme, 0.4% cellulose) as antagonists followed by a 6 and 16 hours incubation in the light. Total RNA was extracted and the expression of chaperones was determined in relation to PR1. The results confirm that cell wall-degrading enzymes inhibit the SA-mediated PR1 expression (FIG. 9). Quantification via PhosphoImaging reveals that at 16 hours of incubation only 36% of the signal is detected in the presence of cell wall-degrading enzymes, which corresponds well with previous findings (Vidal et al, 1997). In contrast, BiP expression is not antagonised by the cell wall-degrading enzymes and appears even to be induced cooperatively after prolonged incubations (16 hours). Obviously, cell wall-degrading enzymes do not have a inhibitory effect on the SA-mediated BiP expression. This suggests that BiP is controlled by a different SA-dependent regulatory mechanism to PR1.

### EXAMPLE 10

### The unfolded protein response is additive to the SA response of BiP

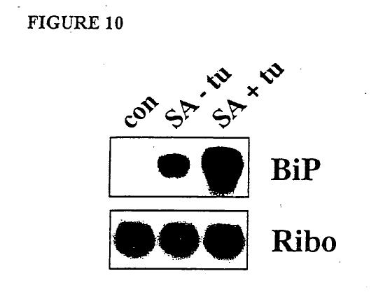

In example 6 we have shown that the UPR-induced BiP gene expression is additive to the cell wall-degrading enzyme (CDE) response. We now tested whether the SA- and UPR-mediated induction of BiP expression were additive as well. For this purpose, plants were sprayed with 5 mM SA and incubated for 12 h. To superimpose the UPR onto the SA response, the SA-treated leaves were transferred to Petridishes which contained MS medium with and without tunicamycin. After floating of the leaves for 2.5 h on this medium, RNA was extracted and BiP gene expression was analysed by Northern blotting. FIG. 10 shows that BiP gene expression is strongly induced in the SA-treated plants (compare lane con with lane SA−tu). In addition, a further induction of BiP transcription is established by tunicamycin treatment (compare lane SA−tu with lane SA+tu). This induction is due to the presence of tunicamycin in the MS medium and is not an artefact due to prolonged floating (2.5 h) of the leaves on the medium as the negative control leaves (SA−tu) have floated as well 2.5 h on MS-medium without tunicamycin. The fact that, upon SA treatment, tunicamycin is still able to induce BiP gene expression shows that both stimuli are additive which suggests that the two induction mechanisms are different. EXAMPLE 11

### Different signal transduction pathway are used for the induction of PR Genes and BiP

To gain further insight into the signal transduction pathways leading to the induction of BiP and PR1, an SA non-responsive mutant of Arabidopsis thaliana (saiI) was used which does not express PR1 in the presence of SA (Shah et al, 1997). WT and saiI mutants of the same ecotype were sprayed with SA (5 mM) and incubated in the light. Total RNA was extracted and probed with the PR1 and BiP gene from Arabidopsis. The induction of PR1 in WT Arabidopsis was detected 3 hours after SA-treatment and continued to increase until 8 hours (FIG. 10). BiP showed the same expression profile as seen in tobacco plants when treated with SA. As in tobacco plants, BiP mRNA levels increase prior to PR1 transcripts in SA treated Arabidopsis plants (FIG. 10) and diminish after prolonged incubations (data not shown). In the Arabidopsis saiI mutant, the PR1 induction was completely abolished during SA-treatment as expected (Shah et al, 1997). In contrast, BiP mRNA levels in the mutants showed exactly the same induction pattern as in the wild-type plant. This demonstrates that either a different SA-dependent signal transduction pathway is used to induce the BiP gene, or that the regulatory protein which is defective in saiI mutants is located downstream of the BiP gene in the signal transduction pathway leading from SA to induced PR1 or BiP gene induction.

### EXAMPLE 12

### Induced BiP levels accelerate the SA-mediated PR1 induction

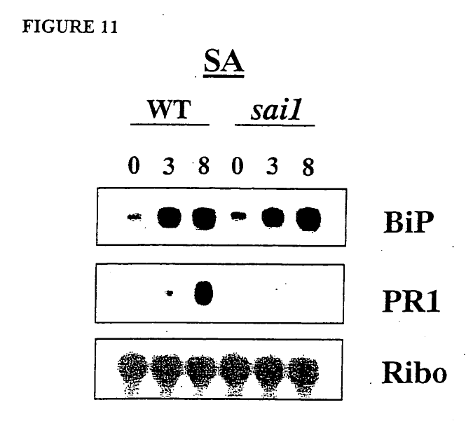

To distinguish between the two possible working models, we tested PR1 gene expression in transgenic plants carrying the BiP coding region under the control of the strong constitutive Cauliflower Mosaic Virus (CaMV) 35S promoter. If BiP is simply located on the signal transduction pathway upstream of the saiI mutation. BiP overproduction alone should lead to induced PR1 gene expression. Transgenic plants which show 5-fold increased BiP steady state protein levels and 142-fold increased BiP transcript levels were used to test basal PR1 mRNA levels and SA-mediated PR1 induction. As shown in FIG. 11, basal BiP mRNA levels are much higher in the BiP overproducing plants The weak induction by SA was unexpected but could be due to the influence of SA on the CaMV35S promoter itself (Qin et al, 1994). FIG. 11 shows that the overproduction of BiP alone does not replace the SA signal because it does not lead to induction of the PR1 gene (compare lanes 0 with each other). However, the BiP overproducing plants show a more rapid PR1 induction upon SA treatment compared to the wild-type plant. Together the results show that high BiP levels in the ER promote the SA-mediated PR1 induction but cannot replace the SA signal. This suggest that a branched signal transduction pathway leads to the induction of BiP and PR1 upon SA treatment and that there is cross-talk between the two branches of the pathway.

### EXAMPLE 13

### Secretory protein synthesis is inhibited by ER stress

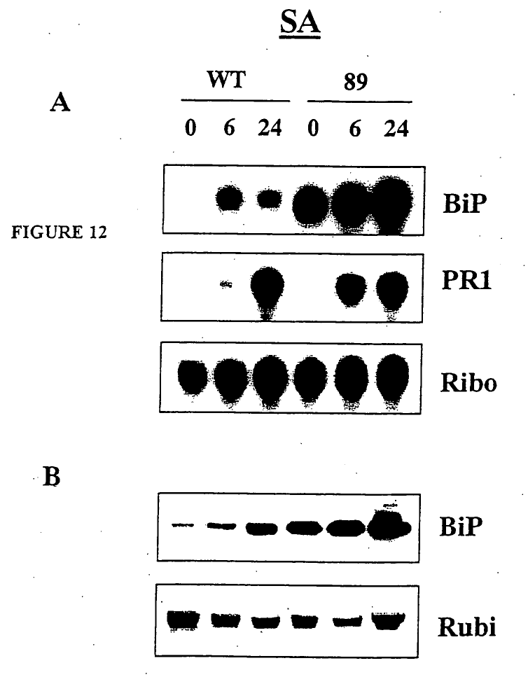

We have established a model system based on the comparison of protein biosynthesis in the cytosol and on the rough ER using transient expression. A plasmid was constructed (pNL200, FIG. 12A) containing two genes, one encoding the secreted barley α-amylase (Rogers, 1985) and the other encoding the cytosolic marker β-glucuronidase (GUS; Jefferson et al., 1987). α-Amylase was used to measure secretory protein biosynthesis, and GUS was used to control for transfection efficiency and overall cell viability. We compared cells under normal culture conditions with cells subjected to ER stress by treatment with tunicamycin. FIG. 12B shows that tunicamycin does not affect cell viability during the course of the experiment, as monitored with the internal marker GUS, confirming previous results (Denecke et al., 1990). In contrast, total α-amylase activity in the cell suspension was greatly reduced. Since α-amylase is not glycosylated, tunicamycin should not have a direct effect on this protein. The tunicamycin effect is protein-specific and not dependent on the promoter used. Therefore, we postulated that during tunicamycin stress, α-amylase synthesis, translocation, or folding is compromised.

### EXAMPLE 14

### Artificially increased BiP levels alleviate ER stress as measured by secretory protein production

One possible explanation for the tunicamycin effect could be that BiP is recruited by other malfolded proteins and is not available in sufficient quantities to promote optimal α-amylase synthesis, translocation, and folding. To test this hypothesis, we coexpressed BiP to determine whether increased BiP levels would alleviate the ER stress and restore efficient α-amylase production.

The protoplasts were coelectroporated with pNL200 and plasmids carrying (1) a gene encoding the bulk flow secretory marker phosphinothricin acetyltransferase (ssPAT; Denecke et al., 1990), (2) a BiP overexpression construct (pDE800), or (3) a BiP antisense construct (pNL100). The BiP isoform used in these experiments was the one that complemented the yeast KAR2 mutant (Denecke et al., 1991), whereas ssPAT is a neutral secretory protein used for control purposes. The protoplasts were incubated for 20 hours with and without tunicamycin and the activities of α-amylase and GUS were measured.

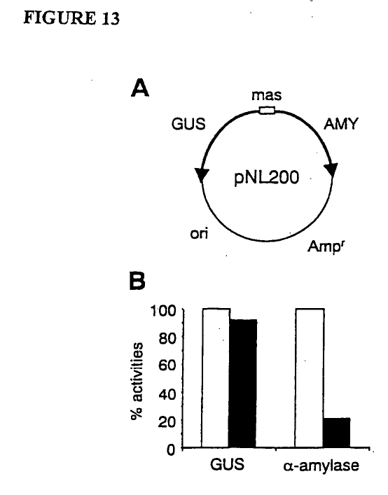

Experiments were conducted in such a manner that similar internal marker activities (GUS) were obtained in each experiment. The α-amylase activity of the total extract was then corrected with the final GUS activities, and FIG. 13A shows the ratio of α-amylase activity to GUS activity. If PAT is coexpressed, tunicamycin leads to a reduction of α-amylase activities, as shown in FIG. 12. BiP coexpression alone leads to slightly lower α-amylase activities compared to PAT coexpression, but no further reduction of α-amylase activity was seen during tunicamycin treatment. Coexpression of the antisense construct was indistinguishable from PAT coexpression. FIG. 13B shows the percentage of α-amylase activity that remains after tunicamycin treatment and illustrates clearly that BiP overexpression protects the cells from tunicamycin stress.

### EXAMPLE 15

### ER stress leads to a reduction of mRNA levels corresponding to genes encoding secretory proteins

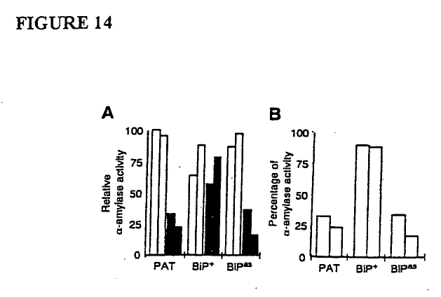

FIG. 14 shows that the transcript level of the vacuolar PR protein β-1,3-glucanase rapidly decreases during UPR-induced BiP transcription but begins to rise again when the BiP mRNA level has reached its maximum. During prolonged incubation times, transcript levels encoding secretory proteins recover to almost normal (initial) values again. Identical results were obtained with other transcripts encoding the secretory proteins acidic chitinase and basic chitinase present in tobacco protoplasts, showing that the effect is not restricted to β-1,3-glucanase (data not shown). The data suggest that inhibition of secretory protein synthesis, observed during ER stress (FIG. 12), occurs prior to translation.

### EXAMPLE 16

### Artificially increased BiP levels alleviate ER stress as measured by mRNA levels corresponding to genes encoding secretory proteins

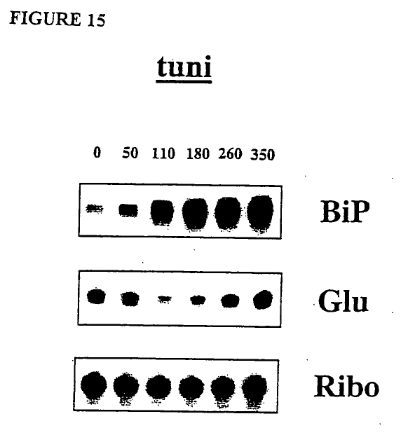

FIG. 15A shows that in BiP overexpressing tobacco protoplasts (89), no tunicamycin-mediated reduction of β-1,3-glucanase mRNA levels is observed, consistent with the hypothesis that the effect is due to limiting amounts of BiP. Overexpression of a BiP derivative lacking the ER retention signal (801) only partially restores the β-1,3-glucanase mRNA level under ER stress conditions (FIG. 15B). This would be expected as the lack of a retention signal will result in a lower BiP level in the ER lumen. The result also suggest that it is the level of BiP in the ER lumen, and not the level of BiP transcripts which is important in this respect

As judged from our present results, two distinct mechanisms operate during a typical UPR. One mechanism is the well established induction of expression of BiP and other ER chaperone genes during accumulation of unfolded proteins in the ER (Shamu, 1997). A second mechanism ensures that secretory protein synthesis is held at a minimum at times when the amount of BiP is limiting. This mechanism is post-transcriptional, and requires information to be present on the transcripts encoding secretory proteins. As soon as BiP transcription is induced and sufficient BiP is being synthesised to replenish the pool, the negative regulation is abolished and efficient secretory protein synthesis is permitted to take place once again.

The proposed mechanism would limit ER stress to a minimum. Clearly, when the amount of BiP is limiting, further protein synthesis on the rough ER would cause additional ER stress. This result could also explain the delay in PR gene induction until sufficient BiP is available.

### MATERIALS AND METHODS

Plant Material and Growth Culture Conditions

Plants of Nicotiana tabacum cultivar Petit Havana (Maliga et al., 1973) were axenically grown in MS medium (Murashige and Skoog, 1962), 2% Sucrose in a temperature controlled room at 25° C. with a 16 h day/8 h light regime and a light irradiance of 200 μE.m⁻².s⁻¹. Arabidopsis thaliana Nössen WT and saiI plants (Shah et al., 1997) were grown under the same conditions.

SA-Treatment

Plants were sprayed with 5 mM SA and 0.5% Tween 20 from all sides, ensuring contact on both sides of each leaf of the plant. Typically, 10 ml of the SA solution was sprayed onto each plant. Plants were then transferred from the 16 h day/8 h night regime to constant light regime.

Northern Blots

Protoplasts RNAs were extracted as described by Jones et al., (1985). Leaves were ground in frozen liquid nitrogen and transferred to NTES buffer (0.1 M NaCl, 10 mM Tris pH 7.5, 1 mM EDTA, 1% SDS). Protoplasts samples were frozen in liquid nitrogen and thawed in NTES buffer. RNA was extracted after adding an equal volume of phenol/chloroform. Ethanol precipitation was carried out after incubation at −20° C. for 16 hours. The pellet was resuspended and RNA was selectively precipitated with LiCl (2M LiCl) for 2 hours on ice. The pellet was washed with 70% ethanol and resuspended in diethyl pyrocarbonate-treated water. Gel blots of total RNA denatured in formamide and formaldehyde were prepared. RNA was blotted onto Hybond-N membrane (Amersham Corp), as described by the manufacturer. A BLP4 BiP specific probe containing full length cDNA, partial length BiP2 were labelled using random prime DNA synthesis using Klenow fragment of DNA polymerase I. Hybridisation was performed as previously described (Denecke et al., 1995). Probes for tobacco BiP (Denecke et al., 1991), tobacco PR1a (Cornelissen et al., 1986), Arabidopsis BiP and Arabidopsis PR1a were prepared as described (Denecke et al., 1995; Vidal et al., 1997). As a riboprobe, we used the 28S RNA from asparagus, kindly provided by J. Draper, University of Wales, Aberystwyth.

Protein Gel Blotting

Fully expanded leaves were collected and quickly frozen in liquid nitrogen. Frozen samples were then ground with a mortar and pestle. Protein concentrations were determined using Bio-Rad protein assay reagent

Proteins in SDS-polyacrylamide gels were transferred onto a nitrocellulose membrane and then blocked with PBS, 0.5% Tween 20, and 5% milk powder for 1 hr. The filter was then incubated in blocking buffer with primary antibody at a dilution of 1/5000 for anti-BIP and anti-calreticulin antibodies. Antibodies to barley α-amylase were used at a dilution of 1/10000. After 1 hr, a 15-min wash and three 5 min washes were done with 1×PBS and 0.5% Tween 20. The secondary antibody used was anti-rabbit antibody conjugated to horseradish peroxidase at a dilution of 1/5000 in 1×PBS, 0.5% Tween 20, and 5% milk powder. The filter was incubated with the secondary antibody for 1 hr. Washes were for 15 min, with 4 washes of 5 min with 1×PBS and 0.5% Tween 20 followed by a final wash with 1×PBS. Detection of antigen-antibody complexes was performed with enhanced chemiluminescence (ECL, Amersham Corp), and the images were recorded on film.

Plasmid Constructs

All DNA manipulations were done according to established procedures. The Escherichia coli MC1061 ampicillin-resistant strain (Casadaban and Cohen, 1980) was used for the amplification of all plasmids.

Plasmids for Transient Expression

The plasmid pDE203 containing the dual mannopine synthase (mas) promoter driving the chloramphenicol acetyltransferase (CAT) gene and the β-glucuronidase (GUS) gene is identical to pDE222 (Denecke et al., 1992), except for the presence of the CAT coding region rather than the bar coding region. pDE203 was digested with NcoI, filled in using the Klenow fragment of DNA polymerase I, and digested with HindIII. The α-amylase coding region was inserted as a blunt HindIII fragment, resulting in pNL200 (FIG. 7).

The lumenal binding protein (BiP) coding region of isoform BLP4 (Denecke et al., 1991) was amplified by polymerase chain reaction, creating an NcoI site overlapping with the translation initiation codon and a BamHI site just after the stop codon. This fragment was inserted between the cauliflower mosaic virus (CaMV) 35S promoter and the 3′ untranslated end of the nopaline synthase (nos) gene present on pDE4 (Denecke et al., 1990), resulting in pDE800. To obtain a BiP antisense construct, pDE800 was digested with NcoI and BamHI releasing the BiP sequence, the vector was dephosphorylated using calf intestine alkaline phosphatase, and both vector and fragment were filled in. After gel purification, the two parts were ligated again. The plasmid containing the BiP coding region in the antisense orientation was named pNL100.

Plasmids for Stable Expression

Chimeric genes containing the CaMV 35S promoter and the coding region of BiP, in sense and antisense orientations, were ligated into the Agrobacterium tumefaciens transformation vector pDE1001 (Denecke et al., 1992).

Plant Transformations

The pT plasmids were mobilised into the Agrobacterium tumefaciens rifampicin-resistant strain C58 (pGV2260) (Debleare et al., 1985) using the kanamycin resistant E. coli helper strain HB101 (pRK2013). Transformed plants were obtained by agrobacteria infection of leaf pieces with the respective strains. Transformants were selected on Murashige and Skoog medium with 3% sucrose containing 100 μg/mL kanamycin and 250 μg/mL cefotaxin.

Transient Expression Experiments

Tobacco leaf protoplasts (from transformed or untransformed plants) were prepared, and electroporation experiments were performed as previously described (Denecke and Vitale, 1995), with minor modifications to the electroporation conditions. The conditions used in these experiments were 910 μF and 130 V. These optimal conditions were established with the expression of the α-amylase gene in tobacco protoplasts. For each experiment 2.5×10⁶ protoplasts were used with 20 to 40 μg of DNA. After 24 or 48 hr, the protoplasts were analysed by enzymatic assay or by protein gel blotting. Tunicamycin was used at a concentration of 20 μg/mL. Harvesting of cells and culture medium was done as described previously (Denecke and Vitale, 1995).

Enzymatic Assays

α-Amylase activity was measured with a kit (Megazyme, Australia). The reaction was performed in a microtiter plate at 45° C. with 30 μL of extract and 30 μL of substrate. The reaction was stopped by the addition of 150 μL of stop buffer. The absorbance was measured at a wavelength of 405 nm, with a microtiter plate reader against a blank containing stop buffer alone. Each experiment was carried out twice with three replicates.

GUS activity in protoplasts was measured with a calorimetric assay. six mililiters of floated protoplasts were diluted in TEX buffer (B5 salts, 250 mg/L NH₄NO₃, 750 mg/L CaCl₂, 500 mg/L MES, and 0.4 M sucrose, pH 5.7) and were spun down by the addition of 20 mL of 250 mM NaCl. The pellet was resuspended in extraction buffer (50 mM phosphate buffer, pH 7.0, 10 mM Na₂-EDTA, 0.1% sodium lauryl sarcosine, 0.1% Triton X-100, and 10 mM β-mercaptoethanol) and sonicated. The reaction was performed at 37° C. as follows. Five hundred microliters of 2× reaction buffer (50 mM phosphate buffer, pH 7.0, 0.1% Triton X-100, 2 mM PNPG, and 10 mM β mercaptoethanol) was added to 490 μL of dilution buffer (50 mM phosphate buffer pH 7.0, 0.1% Triton X-100, 10 mM β-mercaptoethanol) and 10 μL of supernatant. The reaction was stopped by the addition of 400 μL of 2.5 M 2-amino-2-methyl propanediol. The absorbance was measured at 415 nm against a blank containing stop buffer that had been incubated at 37° C. for the duration of the reaction time.

### REFERENCES

Benhamou, N. (1996). Elicitor-induced plant defence pathways. Trends in Plant Science 1, 233-240
Boller, T. (1991). Ethylene in pathogenesis and disease resistance. In The Plant Hormone Ethylene (ed. A. K. Mattoo & J. C. Suttle), 293-314. Boca Raton: CRC Press
Chen, Z., Silva, H. and Klessig, D. F. (1993). Active oxygen species in the induction of plant systemic acquired resistance by salicylic acid. Science 262, 1883-1886.
Chen, Z. X., Malamy, J., Henning, J., Conrath, U., Sanchezcasa, P., Silva, H., Ricigliano, J. and Klessig, D. F. (1995). Induction modification and tranduction of the salicylic-acid signal in plant defence responses. Proc. Nat. Ac. Sci. U.S.A. 92, 4134-4137.
Cornelissen, B. J. C.; Hooft van Huijsduijnen, R. A. M.; van,Loon, L. C. and Bol, J. F. (1986). Molecular characterization of messenger RNAs for “pathogenesis-related” proteins 1a, 1b and 1c, induced by TMV infection of tobacco. EMBO J. 5, 37-40
Creelman, R. A., Tierney, M. L and Mullet, J. E. (1992). Jasmonic acid/methyl jasmonate accumulate in wound soybean hypocotyls and modulate wound gene expression. Proc. Natl. Acad. sci. USA 89, 4983-4941
Debleare, R., Bytebier, B., De Greve, H., Debroeck, F., Schell, J., Van Montagu, M., and Leemans, J. (1985). Efficient octopine Ti plasmid-derived vectors for Agrobacterium-mediated gene transfer to plants. Nucleic Acids Res. 13, 4777-1788.
Delaney, T., Uknes, S., Vernooij, B. Friedrich, L., Weymann, K, Negrotto, D., Gaffney, T., Gut-Rella, M., Kessman, H., Ward, E and Ryals, J. (1994). A central role of salicylic acid in plant disease resistance. Sciencedd. 266, 1247-1250.
Denecke, J., and Vitale, A. (1995). The use of plant protoplasts to study protein synthesis, quality control, protein modification and transport through the plant endomembrane system. In Methods in Cell Biology, Vol. 50, D. W. Galbraith, H. J. Bohnert, H., and D. P. Bourque, eds (San Diego, Calif.: Academic Press), pp. 335-348.
Denecke, J., Botterman, J., and Debleare, R (1990). Protein secretion in plant cells can occurs via a default pathway. Plant Cell 2, 51-59.
Denecke, J., De Rycke, R., and Botterman, J. (1992). Plant and mammalian sorting signals for protein retention in the endoplasmic reticulum contain a conserved epitope. EMBO J. 11, 2345-2355.
Denecke, J., Goldman, M. H. S., Demolder, J., Seurinck, J., and Botterman, J. (1991). The tobacco luminal binding protein is encoded by a multigene family. Plant Cell 3, 1025-1035
Denecke, J., Carlsson, L. E., Vidal, S., Höglund, A-S, Ek, B., van Zeijl, M. J., Sinjorgo, K. M. C. and Palva, E. T. (1995). The tobacco homolog of mammalian calreticulin is present in protein complexes in vivo. The Plant Cell 7, 391-406.
Durner, J., Shah, J. and Klessig, D. F. (1997). Salicylic acid and disease resistance in plants. Trends in Plant Science 2, 226-273.
Farmer, E. E., Johnson, R. R. and Ryan, C. A. (1992). Regulation of expression of proteinase inhibitor genes by methyl jasmonate and jasmonic acid. Plant Physiol 98, 995-1002
Gaffney, T., Friedrich, L., Vernooij, B., Negrotto, D., Nye, G., Uknes, S., Ward, E., Kessmann, H. and Ryals, J. (1993). Requirement of salicylic acid for the induction of systemic acquired resistance. Science 261, 754-756.
Hyodo, H. (1991). Stress/wound ethylene. In The Plant Hormone Ethylene (ed. A. K. Mattoo & J. C. Suttle), 43-63. Boca Raton: CRC Press
Jefferson, R. A., Kavanagh, T. A., and Michael, W. B. (1987). GUS fusions: β-Glucuronidase as a sensitive and versatile gene fusion marker in higher plants. EMBO J. 6, 3901-3907.
Jones, R. L. (1985). Protein synthesis and secretion by the barley aleurone: a perspective. Israel J Bot 34, 377-395
Kozutsumi, Y., Segal, M., Normington, K, Gething, M.-J. and Sambrook, J. (1988). The presence of malfolded proteins in the endoplasmic reticulum signals the induction of glucose-regulated proteins. Nature 332, 462-464
Leon, J., Lawton, M. A. and Raskin, I. (1995). Hydrogen-peroxide stimulates salicylic-acid biosynthesis in tobacco. Plant Physiology 108, 1673-1678
Malamy, J. and Klessig, D. F. (1992). Salicylic acid and plant disease reisistance. Plant J. 2, 643-654.
Maliga, P., Breznowitz, A., and Marton, L. (1973). Streptomycin-resistant plants from callus culture of haploid tobacco. Nature 244, 29-30.
Mauch, F., Mauch-Mani, B. and Boller, T. (1988). Antifungal hydrolases in pea tissue. II. Inhibition of fungal growth by combinations of chitinase and β-1.3 glucanase. Plant Physiol. 88, 936-942.
Murashige, T. and Skoog, F. (1962). A revised medium for rapid growth and bioassays with tobacco tissue culture. Physiol. Plant. 15, 473-479.
Palva, T. K., Holmström, K.-O., Heino, P., and Palva, E. T. (1993). Induction of plant defense response by exoenzymes of Erwinia crotovora subsp. crotovora Mol. Plant-Microbe Interact. 6, 190-196
Palva, T. K., Hurtig, M., Saindrenan, P., and Palva, E. T. (1994). Salicylic induced resistance to Erwinia crotovora subsp. crotovora in tobacco. Mol. Plant-Microbe Interact. 7, 356-363
Pérombelon, M. C. M. and Salmond, G. P. C. (1995) Bacterial soft rots. In Pathogenesis and host specifity in plant diseases (ed. U S Sing, R P Singh and K kohmoto), 1,1-20. Oxford:Pergamon
Qin, X. F., Holuigue, L., Horvath, D. M. and Chua, N. H. (1994). Immediate-early transcription activation by salicylic-acid via the cauliflowre mosaic-virus as-1 element. Plant Cell 6, 863-874.
Rogers, J. (1985). Two barley α-amylase gene families are regulated differently in barley aleurone cells. J. Biol. Chem. 260, 3731-3738.
Ross, A. F. (1961). Localized acquired resistance to plant virus infection in hypersensitive hosts. Vir 14:329-339
Sela-Buurlage, M. B., Ponstein, A. S., Bres-Vloemans, S. A., Melchers, L. S., van den Elzen, P. J. M. and Cornelissen, B. J. C. (1993) Only specific tobacco (Nicotania tabacum) chitinases and beta-1,3-glucanases exhibit antifungal activity. Plant Phys 101, 857-863
Shah, J., Tsui, F. and Klessig D. F. (1997). Characterization of a alicylic acid-insensitive mutant (sail) of Arabidopsis thaliana, identified in a selective screen utilizing the SA-inducible expression of the tms2 gene. Mol. Plant-Microbe Interact. 10, 69-78.
Shamu, C. (1997). Splicing together the unfolded-protein response. Current Biology 7, R67-70.
Vidal, S., de Leon, I. P., Denecke J. and Palva, E. T. (1997). Salicylic acid and the plant pathogen Erwinia carotovora induce defense genes via antagonistic pathways. Plant Journal 11, 115-123.
Vidal, S., Eriksson, A. R. B., Montesano, M., Denecke, J. and Palva, E. T. (1998) Cell Wall-degrading enzymes from Erwinia carotovora cooperate in the salicylic acid-independent induction of a plant defense response. Mol Plant-Microbe interact 11, 232-32
Vidal, S., Norman, C. and Palva, E. T. Cross-talk between ethylene, jasmonic acid and salicylic acid-dependent signal pathways regulates defense gene expression triggered by the plant pathogen Erwinia carotovora. In preparation
Vitale, A., Ceriotti, A. and Denecke, J. (1993). The role of the endoplasmic reticulum in protein systhesis, modification and intracellular transport. J. Exp. Bot., 44, 1417-1444.
Vogel, J. P, Misra, L. M., and Rose, M. D. (1990). Loss of BiP/GRP78 function blocks translocation of secretory proteins in yeast J. Cell Biol. 110, 1885-1895.
Walther-Larsen, H., Brandt, J., Collinge, D. B. and Thordal-Christensen, H. (1993) A pathogen-induced gene of barley encodes HSP90 homologue showing striking similarity to vertebrate forms resident in the endoplasmic reticulum. Plant Mol Biol 21, 1097-11098
Ward, E. R., Uknes, S. J., Williams, S. C., Dincher, S. S., Wiederhold, D. L., Alexander, D. C., Ahl-Goy, P., Métraux, J.-P. and Ryals. J. A. (1991). Coordinate gene activity in response to agents that induce systemic acquired resistance. Plant Cell 3, 1085-1094.

1. (canceled)

2. A method of increasing the capacity for secretory protein synthesis in a plant, comprising causing the plant to maintain in at least a part of the plant a level of BiP, or a homologue thereof, which is greater than the endogenous level for said plant in non-stressful conditions.

3. The method according to claim 2 of reducing the period within which the plant's natural defence mechanism responds to attack by a plant pathogen.

4. The method according to claim 2 wherein the maintained level of BiP, or a homologue thereof, is at least three times said endogenous level.

5. The method according to claim 4 wherein said maintained level is at least five times said endogenous level.

6. The method according to claim 2 wherein said maintained level is effected by over expression of BiP, or a homologue thereof, by means of a chimeric gene containing a strong constitutive promoter, a coding region for BiP or a homologue thereof and a 3′ untranslated end containing a stop sequence.

7. The method according to claim 2 wherein said maintained level is effected by over expression of calreticulin, or a homologue thereof, by means of a chimeric gene containing a strong constitutive promoter, a coding region for calreticulin or a homologue thereof and a 3′ untranslated end containing a stop sequence.

8. The method according to claim 2 wherein said maintained level is effected by over expression of the ATPase domain of BiP, or a homologue thereof, and an ER retention signal by means of a chimeric gene containing a strong constitutive promoter, a coding region for the ATPase domain of BiP, or a homologue thereof, and for an ER retention signal and a 3′ untranslated end containing a stop sequence.

9. The method according to claim 2 wherein said maintained level is effected by modifying signal transduction pathways leading to BiP induction.

10. The method according to claim 2 wherein the plant is additionally treated with salicylic acid.

11. A modified plant produced by the method of claim 1 which maintains, in at least a part thereof, a level of BiP, or a homologue thereof, at least three times greater than the level maintained in said part by an unmodified plant of the same species in non-stressful conditions.

12. The modified plant according to claim 11 wherein the level of BiP, or a homologue thereof, is at least five times greater than the level maintained by an unmodified plant of the same species in non-stressful conditions.

13. Use of salicylic acid in combination with overexpression of BiP or a homologue thereof to protect a plant against pathogen attack.

14. A modified plant or plant cells produced by the method of claim 2 with a level of BiP, or a homologue thereof, which is at least three times greater than the endogenous level of the plant or plant cells in non-stressful conditions.

15. The modified plant or plant cells according to claim 14 wherein the level of BiP, or a homologue thereof, is at least five times greater than the endogenous level of the plant or plant cells in non-stressful conditions.


SEQUENCE LISTING (2 sequences)

SEQ ID NO: 1 - protein, 4 aa, mammalian
KDEL

SEQ ID NO: 2 - protein, 4 aa, Saccharomyces cerevisiae
HDEL